(19) United States
(12) Patent Application Publication
(10) Pub. No.: US 2022/0220533 A1
(43) Pub. Date: Jul. 14, 2022

(54) METHOD OF DIAGNOSING AND MONITORING SUBSTANCE ADDICTION OR BEHAVIORAL ADDICTION USING C-KIT BIOMARKER

(71) Applicant: Wuhan University, Wuhan (CN)

(72) Inventors: Yanqin LI, Wuhan (CN); Xuechuan HONG, Wuhan (CN); Zilin CHEN, Wuhan (CN); Shimin ZHU, Wuhan (CN); Xinyu ZHANG, Wuhan (CN); Mingzhu CHEN, Wuhan (CN)

(21) Appl. No.: 17/708,005

(22) Filed: Mar. 30, 2022

Related U.S. Application Data
(63) Continuation-in-part of application No. PCT/CN2020/119255, filed on Sep. 30, 2020.

(30) Foreign Application Priority Data
Sep. 30, 2019 (CN) 201910939677.4
Sep. 30, 2019 (CN) 201910939688.2

(51) Int. Cl. C12Q 1/686; A61P 25/36; A61K 31/506

(52) U.S. Cl. CPC C12Q 1/686; A61K 31/506; A61P 25/36

(57) ABSTRACT

A method of diagnosing and monitoring substance addiction or behavioral addiction, the method including using a biomarker. The biomarker is a biomarker produced by c-Kit gene.

### CROSS-REFERENCE TO RELATED APPLICATIONS

This application is a continuation-in-part of International Patent Application No. PCT/CN2020/119255 with an international filing date of Sep. 30, 2020, designating the United States, now pending, and further claims foreign priority benefits to Chinese Patent Application No. 201910939677.4 filed Sep. 30, 2019, and to Chinese Patent Application No. 201910939688.2 filed Sep. 30, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of medicine, and more particularly to a method of diagnosing and monitoring substance addiction or behavioral addiction using a c-Kit biomarker.

Substance addiction and behavioral addiction are serious global public health problems with unclear mechanisms and lack of effective diagnostic and treatment measures. For substance addiction, samples such as saliva and urine are the most routine types of samples used in drug testing, especially in anti-drug and detoxification practice. Due to the relatively short retention period of intermediate metabolites in body fluids after drug use, changes in drug metabolites and limited sensitivity of qualitative detection methods, some illicit drug abusers in the society circumvent the examination by selective temporary suspension, making it impossible to make accurate determination. Similarly, while hair testing can provide information on the long-term drug use of the person being tested, operational measures such as hair care reduce the retention of drugs in the hair, resulting in lower detection rates.

The c-Kit gene, located on human chromosome 4q11-12, is a proto-oncogene and encodes the c-Kit receptor, which is a member of the type III tyrosine kinase receptor protein family. As a ligand for the c-Kit receptor, stem cell factor (SCF), after binding to c-Kit, may activate a variety of downstream signaling pathways, including PI3K/Akt, Ras/ERK, PLC-γ/PKC and other signaling pathways that play an important role in drug reward effects and memory. For example, the phosphorylation activity of c-kit in the nucleus accumbens core was significantly increased after morphine addiction was developed in rats; and imatinib, a c-kit inhibitor, and its analogs improved morphine addiction symptoms by inhibiting c-kit phosphorylation. Recent studies have shown that exosomes, as tiny vesicles with a diameter of 30 to 100 nm and being actively secreted by cells into the extracellular space, exist in large quantities in body fluids such as blood, urine and saliva, and contain proteins, lipids, genetic materials (such as mRNA, miRNA and LncRNA) and other substances, and the exosomes are extremely stable and abundant; similarly, neurons can secrete exosomes, the exosomes in the central nervous system (CNS) can freely cross the human blood-brain barrier (BBB) into the peripheral circulation, and the exosomes in the peripheral circulation can also enter the intracranial cavity through the BBB to exert their effects. The release of exosomes into the circulating blood reflects the functional state of releasing cells, and various contents remain undamaged and exert their corresponding physiological effects, such that the exosomes can be used as circulating biomarkers for disease diagnosis. Therefore, detecting the content state of exosome-derived c-Kit molecules is expected to bring new opportunities for the diagnosis and post-treatment monitoring of substance dependence and behavior dependence.

From a clinical point of view, there are many common important characteristics among various behavioral addictions, such as being unable to control one's own addictive behaviors, taking addictive behaviors as the first need, knowing that it is harmful, but still doing, and even having withdrawal symptoms and tolerance. In particular, “recurrent craving” is the clinical manifestation of the basic pathophysiological mechanism of addiction disorders, and it is also one of the diagnostic criteria for addiction disorders in DSM-5. At present, behavioral addictions such as gambling and Internet addiction have serious negative effects on society, but the mechanism is unclear, there is no clear therapeutic target, and there is a lack of effective drugs. The search for new therapeutic targets is a key issue in the treatment for controlling behavioral addictions.

The c-Kit receptor is one of the tyrosine kinase receptors and is abundantly expressed in addiction-related brain regions. So far, whether c-Kit plays an important role in behavioral addiction and whether it can be used as a target for behavioral addiction treatment have not been reported yet.

### SUMMARY

In response to the problems in the prior art, a first objective of the disclosure is to provide use of c-Kit-related active products as biomarkers for diagnosis and post-treatment monitoring of substance and behavioral addictions.

A second objective of the disclosure is to provide a product for reflecting a substance or behavioral addiction state by tracing or detecting and monitoring an activity of a c-kit gene or a c-kit protein product.

A third objective of the disclosure is to solve problems of increasingly serious behavioral addictions and lack of effective drugs in the prior art, and provide use of c-Kit as a behavioral addiction treatment target in screening drugs or non-drug treatment technologies for behavioral addiction treatment.

According to the disclosure, changes in c-kit phosphorylation levels in brain regions such as nucleus accumbens in rats after acute morphine administration and treatment and with other addiction states are verified by immunohistochemistry and immunofluorescence, molecular biology detection technologies and molecular targeted imaging technologies; a correlation between c-kit mRNA expression quantity in peripheral plasma of a morphine-addicted rat and drug addiction is further analyzed by a real-time quantitative PCR technology; and finally, effects of c-kit inhibitor-imatinib systemic administration on formation and reconsolidation of morphine CPP addiction in mice is investigated through a mouse conditioned place preference (CPP) model. Results show that a c-kit active product can be used as a diagnostic marker to determine a pathological state of drug addiction, and has important application values in anti-drug and detoxification practice as well as other addiction treatments. Through the above detection, use of detecting c-kit-related expression products in preparation of products for addiction diagnosis and post-treatment monitoring is provided. The products include test strips, kits, chips, high-throughput sequencing platforms or imaging detection and other products for detecting c-kit activity. A purpose of detecting activities of c-kit gene, RNA and protein is achieved by these products based on various methods including reverse transcription PCR, fluorescent real-time quantitative PCR, immunoassay, in-situ hybridization, chip, high-throughput sequencing platform or brain functional magnetic resonance and omics.

The addictive substances mentioned in the disclosure refer to narcotic drugs and psychotropic drugs. Among them, the narcotic drugs can be divided into opioids, cocaine and cannabis; the psychotropic drugs are divided into sedative-hypnotics, anxiolytics, central stimulants, hallucinogens and the like; others also include alcohol, tobacco, volatile organic solvents and the like; and addictive behaviors refer to behaviors such as food addiction, Internet addiction, and gambling addiction.

According to the disclosure, high sugar and high fat are administered to SD rats for conditioned place preference modeling, the c-Kit activity changes in addiction-related brain regions are detected by immunohistochemistry, an experiment is conducted by intraperitoneal injection of imatinib mesylate, the conditioned place preference of the experimental rats is observed, and an effect of the drug is analyzed. Results show that after repeated administration of high sugar and high fat, the activity of c-Kit in the addiction-related brain regions is activated, and the intraperitoneal injection of imatinib mesylate inhibits formation of conditioned place preference on high sugar and high fat in rats, and can also block the conditioned place preference reawakened by environmental re-exposure or unconditioned re-exposure, which cannot be relapsed. Administration of imatinib mesylate in nucleus accumbens, a brain region related to reward, can inhibit reawakening of the psychological craving in rats by environmental re-exposure or unconditioned re-exposure after high-sugar and high-fat addiction. The above results show that c-Kit plays an important role in behavioral addiction, and designing drugs for it is expected to abstain from behavioral addiction.

Based on the research according to the disclosure, the disclosure provides the following technical solutions:

use of c-Kit as a therapeutic target for behavioral addiction in screening drugs for treatment of behavioral addiction.

The behavioral addictions include addictive behaviors in gambling, eating, sexual behavior, Internet, work, exercise, spiritual compulsions (e.g., religious devotion) and shopping.

The drugs for the treatment of behavioral addiction are drugs that have an inhibitory effect on c-Kit, such as imatinib or its derivative imatinib mesylate.

Compared to existing addiction diagnosis and treatment detection technologies, the disclosure can achieve the following technical effects:

The disclosure discovers a molecular marker for diagnosing drug addiction, and the use of this molecular marker can determine the early onset of drug addiction and prevent drug users from causing greater harm to themselves or the society; at the same time, it can detect a dynamic pathological change process of addiction for post-treatment monitoring; the disclosure provides a target for etiological maintenance treatment for treating behavioral addiction; and the disclosure is effective and expected to treat behavioral addiction and prevent relapse from the root.

### BRIEF DESCRIPTION OF THE DRAWINGS

FIGS. 1A-1B are graphs showing immunohistochemical results; FIG. 1A is a graph showing c-kit expression in a brain region of an acute morphine rat; FIG. 1B is a graph showing c-kit expression in a brain region of an acute morphine rat after imatinib mesylate administration.

FIG. 2 is a graph showing monitoring and analysis of living brain fluorescent imaging of a near-infrared II region in a rat.

FIGS. 3A-3B are graphs showing detection results of c-kit mRNA expression levels within peripheral plasma exosomes of a morphine-addicted rat; FIG. 3A is a flow chart of an administration experiment; FIG. 3B is a graph showing analysis and detection results of c-kit mRNA within plasma exosomes.

FIGS. 4A-4B are graphs showing effect of imatinib mesylate on formation of morphine addiction in mice; FIG. 4A is a flow chart of an administration experiment;

FIG. 4B shows effect of imatinib mesylate on a CPP score.

FIGS. 5A-5B are graphs showing effect of imatinib mesylate on psychological craving of mice after morphine addiction; FIG. 5A is a flow chart of an administration experiment; FIG. 5B shows effect of imatinib mesylate on a CPP score.

FIGS. 6A-6C are graphs activation of c-Kit activity in addiction-related brain regions after high-sugar and high-fat food administration.

FIG. 7 shows that imatinib mesylate inhibits formation of conditioned place preference on a high-sugar and high-fat diet in rats.

FIGS. 8A-8B show that imatinib mesylate blocks conditioned place preference reawakened by environmental re-exposure or unconditioned re-exposure.

FIGS. 9A-9B show that administration of imatinib mesylate to nucleus accumbens inhibits conditioned place preference reawakened by environmental re-exposure or unconditioned re-exposure.

FIG. 10 shows that gambling behavior induces enhanced c-Kit activity in nucleus accumbens.

FIG. 11 shows that inhibition of c-Kit phosphorylation levels by imatinib mesylate eliminates gambling behavior.

FIGS. 12A-12B show effect of imatinib mesylate on gambling behavior; FIG. 12A: effect of imatinib mesylate on gambling behavior induced by environmental cue, FIG. 12A: effect of direct administration of imatinib mesylate on gambling behavior.

### DETAILED DESCRIPTION

To further illustrate, embodiments detailing a method of diagnosing and monitoring substance addiction or behavioral addiction using a c-Kit biomarker are described below. It should be noted that the following embodiments are intended to describe and not to limit the disclosure.

An opioid used in the following embodiments is morphine. Other opioids have a similar mechanism of action to morphine. Morphine is widely representative, and those skilled in the art can reproduce similar research results in other opioids. Other addictive substances include cocaine, alcohol, nicotine and the like, and addictive behaviors include food addiction and gambling addiction. Materials, reagents and the like used in the following embodiments can be obtained from commercial sources unless otherwise specified.

### Example 1

Effect of acute morphine administration on expression of c-Kit in brain region of rat

1. Materials

Drugs: morphine (Qinghai Pharmaceutical Factory), imatinib mesylate (Selleck Chemicals).

Experimental animals: SPF-grade SD male rats, weighing 180-220 g, were purchased from the Animal Experiment Center of Three Gorges University with an animal qualification certificate number of NO. 42010200001637 and a production license number of SCXK (Hubei) 2017-0012. Rat feed was purchased from the Laboratory Animal Center of Wuhan University.

2. Experimental Method

(1) Immunohistochemical Detection of c-Kit Activity Changes in Addiction-Related Brain Regions and Activation of Signal Transduction Pathways Thereof

Experimental rats were divided into solvent groups (a normal saline+normal saline group, a normal saline+imatinib mesylate group) and morphine groups (a morphine+normal saline group, a morphine+imatinib mesylate group) (n=5). The rats were intraperitoneally administrated with 1 mL/kg of a solvent or 30 mg/kg of imatinib mesylate, respectively. After 30 min, the rats were subcutaneously administrated with 1 mL/kg of normal saline or 10 mg/kg of morphine, respectively. After 1 h, anesthesia and perfusion were performed, a brain tissue was fixed with a 4% paraformaldehyde solution, dehydrated in alcohol solutions with different concentrations, hyalinized in xylene, embedded in paraffin, and then cut into 3 μm-thick sections. Tissues were deparaffinized, boiled in 0.01M sodium citrate buffer (pH 6.0) for 10-15 min, and cooled in cold water to room temperature. Then multi-label immunohistochemistry and immunofluorescence co-localization were performed to detect the changes of the c-Kit activity in the addiction activation-related brain regions, the activation of the signal transduction pathways thereof and a blocking effect of imatinib mesylate, so as to determine c-Kit-specific brain regions for addiction activation and a preventive effect of imatinib mesylate.

(2) Detection of Dynamic Changes of c-Kit Activity in Addiction-Related Brain Regions by Targeted Molecular Imaging Technology

NIR-II fluorescence imaging analysis: the experimental rats were randomly divided into a solvent group, an acute morphine group and an acute morphine+imatinib mesylate group (n=5). The rats were intraperitoneally administrated with 1 mL/kg of normal saline or 30 mg/kg of imatinib mesylate, respectively. After 30 min, the experimental rats were subcutaneously administrated with 1 mL/kg of normal saline or 10 mg/kg of morphine, and then the experimental rats were given 100 μg of PEG1000-c-Kit fluorescent probe by tail vein administration. Under the guidance of NIR-II fluorescence imaging, dynamic changes of the brain regions of the rats were dynamically monitored and photographed at 2 h, 6 h, 8 h and 12 h with a near-infrared II region imaging camera, respectively, so as to indicate changes in a phosphorylation level of c-Kit protein. After detection at 12 h, the rats were deeply anesthetized and decapitated to collect brains, the brain regions were further located and the changes of the c-Kit activity in the brain were clearly observed. The c-Kit-specific brain regions for addiction activation and a monitoring effect of the dynamic changes of the c-Kit activity after imatinib mesylate treatment were further determined.

3. Experimental Results

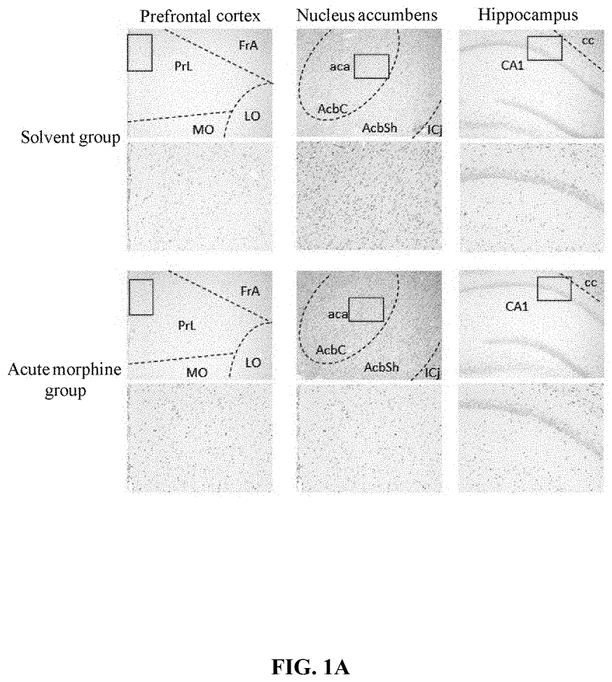

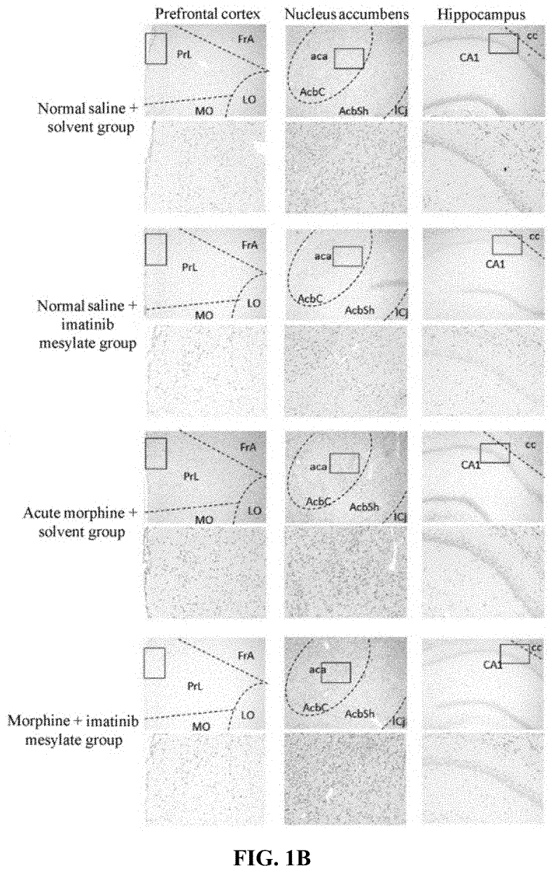

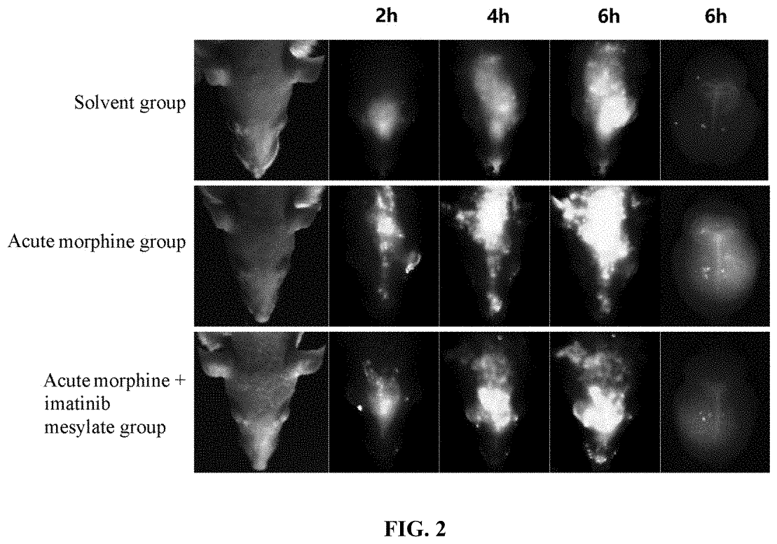

Immunohistochemical results are shown in FIGS. 1A-1B. Compared with normal rats, the phosphorylation level of c-Kit protein in nucleus accumbens regions of the rats was significantly increased 1 h after acute morphine administration, and there was no significant change in prefrontal cortex, hippocampus and other regions (FIG. 1A); after the rats were given imatinib mesylate, the phosphorylation activity of c-Kit protein in the brain regions such as nucleus accumbens, prefrontal cortex and hippocampus was significantly decreased (FIG. 1B). MR fluorescence imaging detection also found that brain brightness of the rats after acute morphine administration became brighter and brighter, and an enrichment intensity of the probes reached a peak after 6 h, which was in obvious contrast with the normal saline group, indicating that acute morphine administration could significantly enhance the phosphorylation activity of c-Kit in the brains of the rats, and after injection of imatinib mesylate serving as a c-Kit inhibitor, the brain brightness of the rats was reduced, and the phosphorylation activity of c-Kit protein activated by morphine was inhibited (FIG. 2).

### Example 2

Changes in Expression Level of c-Kit mRNA within Plasma Exosomes of Morphine-Addicted Rats

1. Materials

Drugs: Morphine (Qinghai Pharmaceutical Factory).

Experimental animals: SPF-grade SD male rats, weighing 180-220 g, were purchased from the Animal Experiment Center of Three Gorges University with an animal qualification certificate number of NO. 42010200001637 and a production license number of SCXK (Hubei) 2017-0012. Rat feed was purchased from the Laboratory Animal Center of Wuhan University.

2. Experimental Method

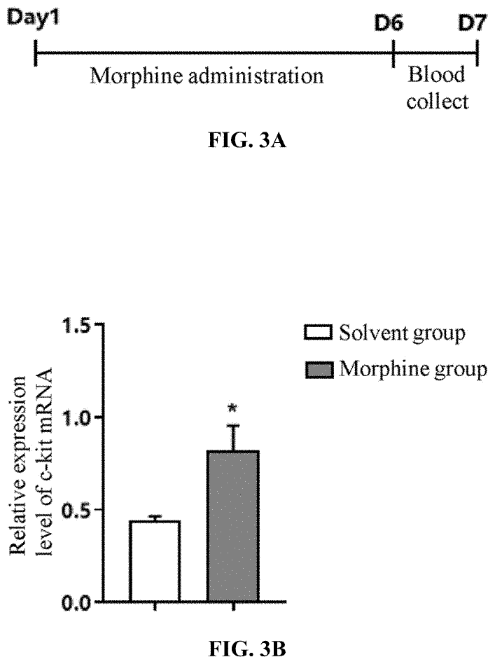

The experimental rats were randomly divided into two groups, a solvent group and a morphine group (n=5). An experimental flow of drug administration is shown in FIG. 3A. The morphine group was subcutaneously administered with 5, 10, 15, 20, 25 and 25 mg/kg of morphine for 6 consecutive days, respectively, and the normal saline group was subcutaneously injected with 1 mL/kg of normal saline every day. After 24 h, eyeballs of the rats were removed and 4 mL of blood was collected.

Plasma separation: after blood collection, whole blood was transferred to a clean 1.5 mL centrifuge tube (containing 140 μL of 50 mM EDTA-Na₂), immediately centrifuged at 1500 rpm for 15 min at 4° C., and supernatant was extracted; then centrifugation was performed again at 3000 rpm for 10 min, and supernatant was extracted.

Exosome Extraction:

(1) 4 mL of PBS solution pre-cooled at 4° C. was added to 1 mL of the centrifuged supernatant of each sample for dilution, and then 1 mL of BPS reagent (Blood PureExo Solution) was added and mixed well.

(2) After standing for 2 h, the mixture was centrifuged at 10000 rpm for 60 min at 4° C., supernatant was discarded, and precipitates were rich in exosome particles. The centrifuged precipitates were evenly pipetted through 400 μL of PBS solution, transferred to a new 1.5 mL centrifuge tube, then centrifuged at 12000 rpm for 2 min at 4° C. or below, and supernatant rich in the exosome particles was retained.

(3) The supernatant was added into an EPF column (Exosome Purafication Filter) and centrifuged at 3000 rpm for 10 min at 4° C. Liquid collected at a bottom of the EPF column was the purified exosome particles.

Exosomal RNA Extraction:

(1) 200 μL of chloroform and 1000 μL of Trizol lysis solution were added to the extracted exosomes as described above, shaken vigorously for 15 s, allowed to stand for 8 min at room temperature, and then centrifuged at 12000 rpm for 15 min at 4° C.

(2) An upper aqueous phase was transferred to a new 1.5 mL Eppendorf tube, an equal volume of isopropanol was added, carefully inverted and mixed, allowed to stand and precipitate in a refrigerator at 4° C. for 10 min, and then centrifuged at 12000 rpm for 10 min at 4° C., and white precipitates at a bottom were retained.

(3) The precipitates were washed by adding 1 mL of 75% ethanol freshly prepared with DEPC-treated water, centrifuged at 10000 rpm for 5 min at 4° C., and then centrifuged at 10000 rpm for 5 min at 4° C., the precipitates were retained and dried for 5 min.

(4) 20 μL of Nuclease-free water was added to the dried precipitates to dissolve RNA, an exosomal RNA solution was obtained, and a concentration of the extracted RNA was determined by UV analysis.

Exosomal RNA Reverse Transcription:

(1) Taking a 20 μL reaction system as an example, each component was added as listed in the table below to obtain a mixture, which was mixed well with the RNA solution, and incubated at 42° C. for 5 min;

Reverse Transcription PCR System:

| Reagent | Usage amount (μL) |
|---|---|
| 5XPrime Script Buffer (for Real Time) | 4 |
| Prime Script RT Enzyme Mix I | 1 |
| RT Primer Mix | 1 |
| Total RNA/Control sample | 10 |
| RNase Free dH2O | 4 |
| Total | 20 μL |

(2) 15 U of AMV reverse transcriptase was added, mixed well and reacted at 37° C. for 15 min, then the sample was boiled at 85° C. for 5 s to inactivate the AMV reverse transcriptase, and the reaction was terminated and placed under an ice bath for 5 min. At this time, a first-strand cDNA was obtained and stored at 4° C.

Real-Time Quantitative PCR Amplification:

(1) Operations were performed on an ABI stepone plus real-time fluorescence quantitative PCR instrument with a Premix Ex Taq™ kit (TakaRa) using the above cDNA as a template.

An upstream primer of c-Kit mRNA was 5′-cgcagcttccttatga ccac-3′ (SEQ ID NO: 1), and a downstream primer was 5′-agtggcctcaactaccttcc-3′ (SEQ ID NO: 2).

An upstream primer for fluorescent quantitative detection of an mRNA expression level of an internal reference gene GAPDH was 5′-ttcaacggcacagtcaagg-3′ (SEQ ID NO: 3), and a downstream primer was 5′-ctcagcaccagcatcacc-3′ (SEQ ID NO: 4).

(2) A reaction system was:

| Reagent | Volume (μL) |
|---|---|
| cDNA template | 2 |
| Upstream primer | 0.8 |
| Downstream primer | 0.8 |
| SYBR Premix Ex TaqII (2x) | 10 |
| ROX Reference Dye (50x) | 0.4 |
| Deionized water | 6 |
| Total volume | 20 |

(3) A procedure for fluorescent PCR amplification was:

| | Temperature (° C.) | Time (s) |
|---|---|---|
| | 95 | 30 |
| 40 cycles | 95 | 5 |
| | 60 | 31 |
| Melting curve | 95 | 30 |
| | 60 | 15 |
| | 95 | 30 |

(4) Each sample was repeated three times and averaged to ensure the accuracy of quantification.

Statistical analysis: with GAPDH as the internal reference gene, c-Kit mRNA was normalized to ensure that the expression level of c-Kit mRNA was compared in an equal number of samples, the relative expression level of c-Kit mRNA=2^{-ΔΔCt}, where ΔΔCt=mean ΔCt value of c-Kit mRNA within plasma exosomes of morphine-addicted rats−(Ct value of c-Kit mRNA within plasma exosomes of morphine-addicted rats−Ct value of GAPDH gene within plasma exosomes of morphine-addicted rats), and a fold change of the expression level of c-Kit mRNA relative to the expression level of the internal reference gene was obtained.

3. Experimental Results

Results are shown in FIG. 3B. Compared with non-addicted rats, the expression level of c-Kit mRNA in peripheral plasma of the morphine-addicted rats was significantly increased, and a difference was statistically significant (p<0.05).

### Example 3

Effect of Imatinib Mesylate on Formation of Morphine Addiction in Mice

1. Materials

Drugs: morphine (Qinghai Pharmaceutical Factory); imatinib mesylate (Selleck Chemicals).

Experimental animals: SPF-grade Kunming strain male mice, weighing 18-22 g, were purchased from the Animal Experiment Center of Three Gorges University with an animal qualification certificate number of NO. 42010200001676 and a production license number of SCXK (Hubei) 2017-0012. Rat feed was purchased from the Laboratory Animal Center of Wuhan University.

Experimental apparatus: Conditional place preference instrument (developed by Institute of Materia Medica, Chinese Academy of Medical Sciences): the experiment was automatically controlled by a computer. The device was a conditioned place preference box consisting of three boxes: two side chambers and one middle chamber. The three chambers were separated by removable partitions, were black both inside and outside. Among them, box A and box B were located on two sides of the middle box, and had the same size. There were 9 squares formed by yellow light-emitting diodes on a side wall of box A, a bottom plate of box A was a stainless steel strip, and a bottom plate of box B was a stainless steel grid. The dwell time and number of entries and exits of the rats in each box could be transmitted to the computer via data, and behavioral information could be collected and recorded automatically.

2. Experimental Method

Animal grouping and treatment: mice were randomly divided into four groups, namely control groups: 1) a normal saline+solvent group, 2) a normal saline+imatinib mesylate administration group; and experimental groups: 3) a morphine+solvent group, 4) a morphine+imatinib mesylate administration group (n=10).

Establishment of Morphine CPP Model

Basal value test: on day 1, the partitions were removed, channels of the three boxes were opened, and a CPP program on the computer was started. The mice were put in from the middle chamber and allowed to move freely in the three boxes for 15 min. The time they stayed in each chamber was recorded via the computer synchronously. According to test results, the mice were eliminated, grouped, and a drug-paired side and a non drug-paired side of each mouse were distinguished.

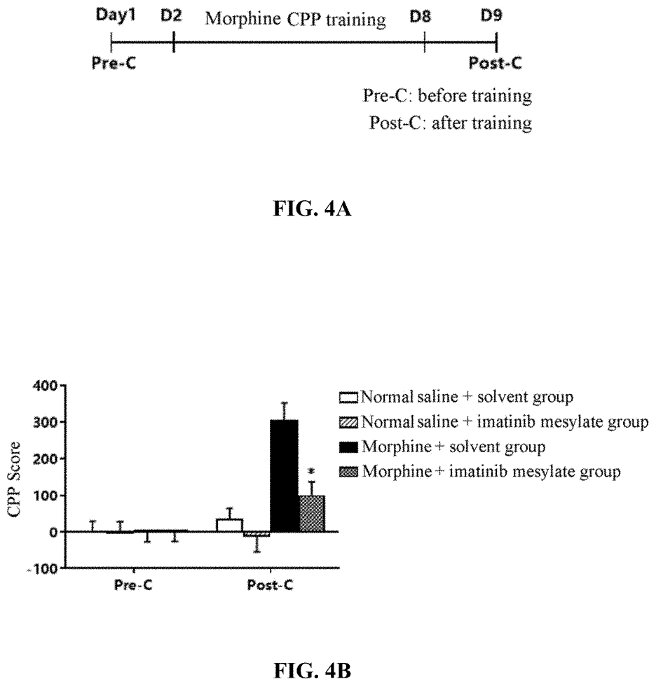

Conditioned place preference training: a schematic diagram of the training was shown in FIG. 4A. From days 2 to 9, the channels among the three boxes were closed. On days 2, 4, 6 and 8, the experimental groups were subcutaneously injected with morphine (15 mg/kg) and placed on the drug-paired side for 45 min, in which the experimental group 3) was intraperitoneally injected with normal saline (1 mL/kg) 30 min before the injection of morphine, and the experimental group 4) was intraperitoneally injected with imatinib mesylate (45 mg/kg). The control groups were subcutaneously injected with normal saline (1 mL/kg) and placed on the non drug-paired side for 45 min, in which the control group 1) was intraperitoneally injected with normal saline (1 mL/kg) 30 min before the injection of morphine, and the control group 2) was intraperitoneally injected with imatinib mesylate (45 mg/kg). On days 3, 5, 7 and 9, the mice in the experimental groups and the control groups were subcutaneously injected with normal saline. The experimental groups were placed on the non drug-paired side, and the control groups were placed on the drug-paired side, both for 45 min. The drug-paired side of each mouse was fixed, and the mice were returned to a rearing cage after each day's training.

Morphine CPP test: on day 10, CPP test, similar to the basal value test phase. The channels among the three boxes were opened without any injection, and the CPP program on the computer was started. The mice were put in from the middle chamber and allowed to move freely in the boxes for 15 min. The time they stayed in each chamber was recorded via the computer synchronously. A CPP score was defined as a difference value between the time spent in the drug-paired chamber and the time spent in the non drug-paired chamber. Whether the mouse developed CPP was determined by comparing a post-measurement value with a pre-measurement value for CPP of the mouse in the drug-paired box.

3. Experimental Results

Results are shown in FIG. 4B. Compared with mice not injected with imatinib mesylate, the conditioned place preference in mice injected with imatinib mesylate could not be formed normally, indicating that imatinib mesylate could prevent morphine addiction by inhibiting c-Kit phosphorylation activity.

### Example 4

Effect of Imatinib Mesylate on Latent Psychological Craving in Morphine-Addicted Mice

1. Materials

Drugs: morphine (Qinghai Pharmaceutical Factory); imatinib mesylate (Selleck Chemicals).

Experimental animals: SPF-grade Kunming strain male mice, weighing 18-22 g, were purchased from the Animal Experiment Center of Three Gorges University with an animal qualification certificate number of NO. 42010200001676 and a production license number of SCXK (Hubei) 2017-0012. Rat feed was purchased from the Laboratory Animal Center of Wuhan University.

Experimental apparatus: Conditional place preference instrument (developed by Institute of Materia Medica, Chinese Academy of Medical Sciences): the experiment was automatically controlled by a computer. The device was a conditioned place preference box consisting of three boxes: two side chambers and one middle chamber. The three chambers were separated by removable partitions, were black both inside and outside. Among them, box A and box B were located on two sides of the middle box, and had the same size. There were 9 squares formed by yellow light-emitting diodes on a side wall of box A, a bottom plate of box A was a stainless steel strip, and a bottom plate of box B was a stainless steel grid. The dwell time and number of entries and exits of the mice in each box could be transmitted to the computer via data, and behavioral information could be collected and recorded automatically.

2. Experimental Method

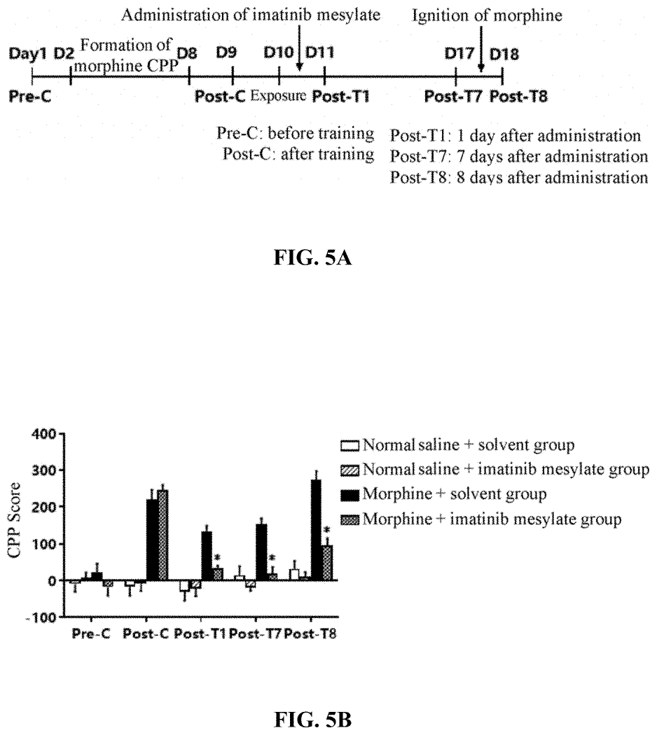

Animal grouping and treatment: mice were randomly divided into four groups: a saline+solvent group, a saline+imatinib sulfonate administration group, a morphine+solvent group, and a morphine+imatinib mesylate administration group, respectively. An experimental flow of drug administration is shown in FIG. 5A.

(1) Establishment of Morphine CPP Model

Basal value test: on day 1, the partitions were removed, channels of the three boxes were opened, and a CPP program on the computer was started. The mice were put in from the middle chamber and allowed to move freely in the three boxes for 15 min. The time they stayed in each chamber was recorded via the computer synchronously. According to test results, the mice were eliminated, grouped, and a drug-paired side and a non drug-paired side of each mouse were distinguished.

Conditioned place preference training: on days 2 to 9, the channels among the three boxes were closed. On days 2, 4, 6 and 8, the experimental groups were subcutaneously injected with morphine (10 mg/kg) and placed on the drug-paired side for 45 min; the control groups were subcutaneously injected with normal saline (1 mL/kg) and placed on the non drug-paired side for 45 min. On days 3, 5, 7 and 9, the mice in the experimental groups and the control groups were subcutaneously injected with normal saline. The experimental groups were placed on the non drug-paired side, and the control groups were placed on the drug-paired side, both for 45 min. The drug-paired side of each mouse was fixed, and the mice were returned to a rearing cage after each day's training.

Morphine CPP test: on day 10, CPP test, similar to the basal value test phase. The channels among the three boxes were opened without any injection, and the CPP program on the computer was started. The mice were put in from the middle chamber and allowed to move freely in the boxes for 15 min. The time they stayed in each chamber was recorded via the computer synchronously. A CPP score was defined as a difference value between the time spent in the drug-paired chamber and the time spent in the non drug-paired chamber. Whether the mouse developed CPP was determined by comparing a post-measurement value with a pre-measurement value for CPP of the mouse in the drug-paired box.

(2) Establishment of a Model for Drug-Seeking Behavior Induced by Environmental Cues

On day 10 of the experiment, mice in each group were intraperitoneally administrated with imatinib mesylate (45 mg/kg) and normal saline (1 mL/kg). After 30 min, the mice in each group were exposed to the drug-paired box, respectively, stayed for 15 min, and then returned to the cage environment.

(3) Morphine CPP Retest

On days 12 and 18, a preference degree of the mice to the drug-paired box was tested, which was similar to the basal value test phase, and on days 13-17 therebetween, no treatment was performed.

(4) Ignition of Morphine CPP

On day 17, ignition was performed with a small dose of morphine (5 mg/kg, i.p.). 5 min after morphine injection, the mice were placed in the middle box and a 15 min CPP value test was started.

3. Experimental Results

Results are shown in FIG. 5B. After the conditioned place preference of mice was formed, the CPP Score was detected after giving normal saline and imatinib mesylate before re-exposure to environmental cues. It was found that the conditioned place preference still existed in the mice given with normal saline, while the CPP Score of the mice given with imatinib mesylate was significantly reduced, the psychological craving caused by the administration environment was suppressed, and was not ignited after 1 week. A difference between the administration group and the control group was significant, indicating that the inhibition of c-Kit could significantly improve the symptoms of morphine addiction.

Conclusions: it can be clearly seen from immunohistochemistry and NIR-II fluorescence imaging that the c-Kit protein phosphorylation expression levels in the nucleus accumbens region of rat brain were enhanced after acute morphine administration, and c-Kit mRNA expression levels in peripheral plasma were also significantly increased after morphine addiction was developed in rats. Imatinib mesylate could significantly inhibit c-Kit activity to prevent and treat addiction and prevent re-ignition. When c-Kit phosphorylation activity was inhibited, morphine addiction in mice could not be formed, and relapse after morphine withdrawal was also significantly inhibited. The above results indicate that c-Kit can be used as a diagnostic biomarker for addiction, and imatinib mesylate can inhibit formation of addiction memory, memory reconsolidation and relapse after withdrawal caused by morphine through inhibiting the phosphorylation activity of c-Kit protein, indicating that after activation of c-Kit caused by addiction, its related products, including proteins, nucleic acids and the like, can be used as diagnostic markers for diagnosing addiction and monitoring its therapeutic effect, which are of great significance in the future of detoxification and anti-drug as well as other types of addiction treatment.

High-sugar and high-fat food used in the following embodiments has similar mechanisms of action in behavioral addictions such as food, and is widely representative. Those skilled in the art can reproduce similar findings in other food addictions or behavioral addictions. Since there are currently no suitable animal models for other types of behavioral addictions such as Internet addiction and gambling addiction, and the mechanism is similar to that of food addiction, an animal model verification is no longer performed herein.

Materials, reagents and the like used in the following embodiments can be obtained from commercial sources unless otherwise specified.

### Example 5

Activation of c-Kit Activity in Addiction-Related Brain Regions by High-Sugar and High-Fat Food

This experiment used homemade high-sugar and high-fat food (40 g original potato chips, 130 g original chocolate cookies, 130 g peanut butter, 130 g chocolate powder seasoning, 200 g powdered laboratory feed and 180 mL water, prepared by mixing in a food processor). The homemade high-sugar high-fat food was rich in sugar, salt and fat (19.6% fat, 14% protein, 58% carbohydrate, 4.5 kcal/g).

After giving rats high-sugar and high-fat food (ad libitum) for 60 min, changes of c-Kit activity in mesolimbic dopamine system including VTA, nucleus accumbens, amygdala, hippocampus and prefrontal cortex were observed by immunohistochemistry combined with western-blot, distribution of activated cells was observed by immunofluorescence co-labeling, and a new molecular mechanism of high-sugar and high-fat food addiction was established.

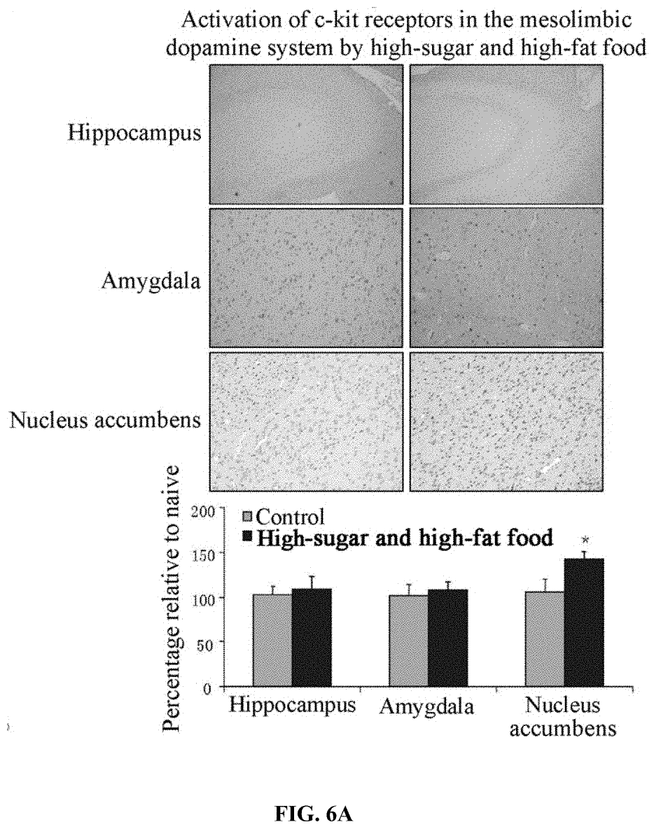

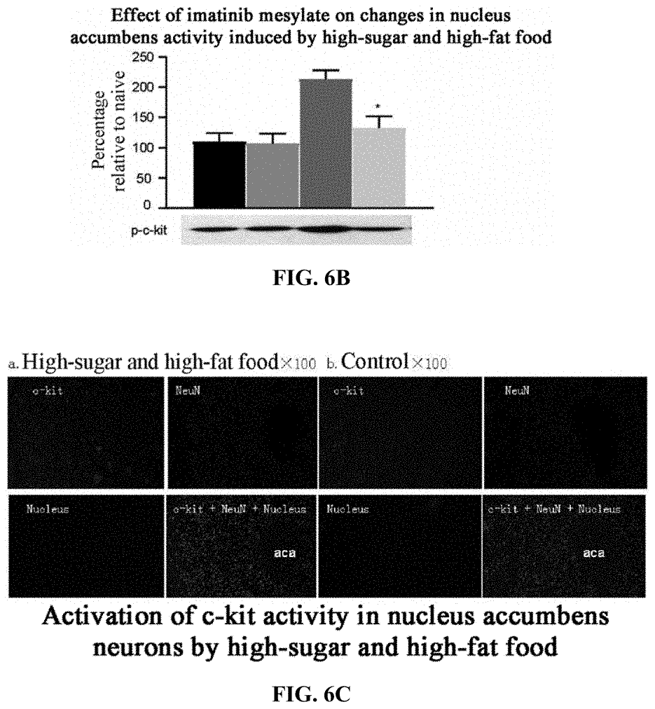

Experimental results showed that high-sugar and high-fat food activated c-Kit receptors in nucleus accumbens neurons, as shown in FIGS. 6A-6C.

### Example 6

Inhibition of Formation of Conditioned Place Preference on High-Sugar and High-Fat Food in Rats by Imatinib Mesylate

In this example, imatinib mesylate was selected as a drug against high-sugar and high-fat food addiction, a conditioned place preference (CPP) model was established for high-sugar and high-fat food, and an effect of imatinib on reward memory of high-sugar and high-fat food was studied. An aim of this study was to determine the role of c-Kit receptors as drug targets in high-sugar and high-fat food addiction, and to select a drug with definite efficacy and low toxicity for the treatment of high-sugar and high-fat food addiction.

1. Materials and Methods

Drugs and reagents: homemade high-sugar and high-fat food (ditto); imatinib mesylate (Novartis PharmaStein AG).

Experimental animals: SPF-grade SD male rats, weighing 220-250 g. The animals were provided by Hubei Provincial Laboratory Animal Research Center with an animal qualification certificate number of NO. 42000600012016 and a production license number of SCXK (Hubei) 2015-2018. Rat feed was purchased from the Laboratory Animal Center of Wuhan University.

Experimental apparatus: Conditional place preference instrument (developed by Institute of Materia Medica, Chinese Academy of Medical Sciences): the experiment was automatically controlled by a computer. The device was a conditioned place preference box consisting of three boxes: two side chambers and one middle chamber. The three chambers were separated by removable partitions, were black both inside and outside. Among them, box A and box B were located on two sides of the middle box, and had the same size. There were 9 squares formed by yellow light-emitting diodes on a side wall of box A, a bottom plate of box A was a stainless steel strip, and a bottom plate of box B was a stainless steel grid. The dwell time and number of entries and exits of the rats in each box could be transmitted to the computer via data, and behavioral information could be collected and recorded automatically.

Experimental Method: Establishment of CPP Model for High-Sugar and High-Fat Food

Basal value test: on day 1, channels among the three boxes were opened, and a CPP program on the computer was started. The rats were put in from the middle chamber and allowed to move freely in the three boxes for 15 min. The time they stayed in each chamber was recorded via the computer synchronously.

Conditioned place preference training: on days 2 to 9, the channels among the three boxes were closed. On days 2, 4, 6 and 8, the experimental groups were intraperitoneally injected with different doses of imatinib mesylate (1, 5, 10, 20 and 30 mg/kg) before ad libitum, and placed on the drug-paired side for 45 min; the control groups were given clear water and placed on the non drug-paired side for 45 min. On days 3, 5, 7 and 9, the rats in the experimental groups and the control groups were all given clear water, the experimental groups were placed on the non drug-paired side, and the control groups were placed on the drug-paired side, both for 45 min. The drug-paired side for each rat was fixed. Each group of rats was then returned to a rearing cage.

CPP test: CPP test was performed on day 10, which was similar to the basal value test phase. The channels among the three boxes were opened without any treatment, and the CPP program on the computer was started. The rats were put in from the middle chamber and allowed to move freely in the three boxes for 15 min. The time they stayed in each chamber was recorded via the computer synchronously. A CPP score was defined as a difference value between the time spent in the drug-paired chamber and the time spent in the non drug-paired chamber. Whether the rat developed CPP was determined by comparing a post-measurement value with a pre-measurement value for CPP of the rat in the drug-paired box. According to the post-measurement value for CPP, the rats that did not form CPP were excluded and the animals were matched and grouped.

Detection indicators: after the rats were trained, the conditioned place preference box was used to detect the addiction of high-sugar and high-fat food. A conditioned place preference score (CPP Score) reflects formation of the rat's addictive behavior. The increase of CPP Score indicates the formation of addictive behavior.

2. Experimental Results

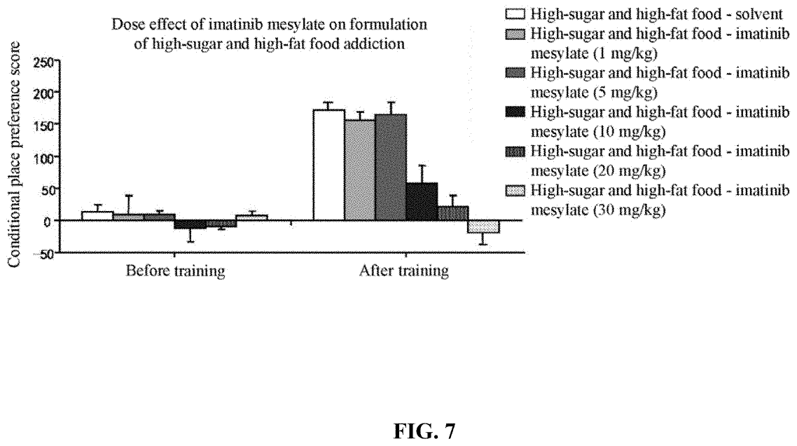

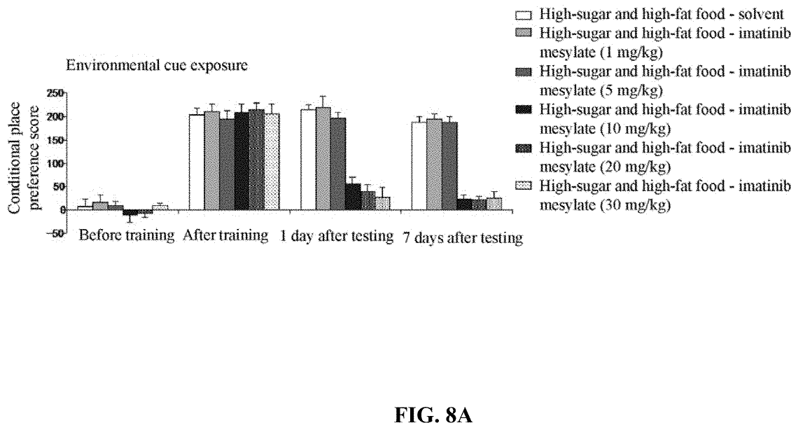

Results showed that the conditioned place preference was formed in the unmedicated rats; the formation of conditioned place preference was inhibited after imatinib mesylate treatment. The results are shown in FIG. 7. Differences among the different dose administration groups with the control group were significant, indicating that c-Kit receptors could be used as a therapeutic target in addiction, and imatinib mesylate had the effect of inhibiting high-sugar and high-fat food addiction.

### Example 7

Blockade of Conditioned Place Preference on High-Sugar and High-Fat Food Due to Environmental Re-Exposure or Unconditioned Re-Exposure by Imatinib Mesylate

In this example, imatinib mesylate was selected as a drug against high-sugar and high-fat food addiction, a conditioned place preference (CPP) model was established for high-sugar and high-fat food, and blockade of conditioned place preference on reward memory of high-sugar and high-fat food due to environmental re-exposure or unconditioned re-exposure by imatinib mesylate was studied; and a drug with definite efficacy and low toxicity for the treatment of high-sugar and high-fat food addiction could be selected.

1. Materials and Methods

Drugs and reagents: homemade high-sugar and high-fat food; imatinib mesylate (Novartis PharmaStein AG).

Experimental animals: SPF-grade SD male rats, weighing 220-250 g. The animals were provided by Hubei Provincial Laboratory Animal Research Center with an animal qualification certificate number of NO. 42000600012016 and a production license number of SCXK (Hubei) 2015-2018. Rat feed was purchased from the Laboratory Animal Center of Wuhan University.

Experimental apparatus: Conditional place preference instrument (developed by Institute of Materia Medica, Chinese Academy of Medical Sciences): the experiment was automatically controlled by a computer. The device was a conditioned place preference box consisting of three boxes: two side chambers and one middle chamber. The three chambers were separated by removable partitions, were black both inside and outside. Among them, box A and box B were located on two sides of the middle box, and had the same size. There were 9 squares formed by yellow light-emitting diodes on a side wall of box A, a bottom plate of box A was a stainless steel strip, and a bottom plate of box B was a stainless steel grid. The dwell time and number of entries and exits of the rats in each box could be transmitted to the computer via data, and behavioral information could be collected and recorded automatically.

Experimental Method

(1) Establishment of CPP Model for High-Sugar and High-Fat Food

Basal value test: on day 1, channels among the three boxes were opened, and a CPP program on the computer was started. The rats were put in from the middle chamber and allowed to move freely in the three boxes for 15 min. The time they stayed in each chamber was recorded via the computer synchronously.

Conditioned place preference training: on days 2 to 9, the channels among the three boxes were closed. On days 2, 4, 6 and 8, the experimental groups were given high-sugar and high-fat food freely and placed on the drug-paired side for 45 min; and the control groups were given cleared water and placed on the non drug-paired side for 45 min. On days 3, 5, 7 and 9, the rats in the experimental groups and the control groups were all given clear water, the experimental groups were placed on the non drug-paired side, and the control groups were placed on the drug-paired side, both for 45 min. The drug-paired side for each rat was fixed. Each group of rats was then returned to a rearing cage.

CPP test: CPP test was performed on day 10, which was similar to the basal value test phase. The channels among the three boxes were opened without any treatment, and the CPP program on the computer was started. The rats were put in from the middle chamber and allowed to move freely in the three boxes for 15 min. The time they stayed in each chamber was recorded via the computer synchronously. A CPP score was defined as a difference value between the time spent in the drug-paired chamber and the time spent in the non drug-paired chamber. Whether the rat developed CPP was determined by comparing a post-measurement value with a pre-measurement value for CPP of the rat in the drug-paired box. According to the post-measurement value for CPP, the rats that did not form CPP were excluded and the animals were matched and grouped.

(2) Establishment of a Model for Drug-Seeking Behavior Induced by Environmental Cues or Unconditioned Re-Exposure

On day 11 of the experiment, after being exposed to the dosing box or given a small amount of high-sugar and high-fat food, the rats were intraperitoneally injected with imatinib mesylate (1, 5, 10, 20 and 30 mg/kg), and then returned to the cage environment.

(3) CPP Retest

On the first and seventh days after the administration of imatinib mesylate, that is, days 12 and 18 of the experiment, a preference degree of the rats to the drug-paired box was tested for 15 min, which was similar to the basal value test phase. On days 13-17 therebetween, no treatment was given to the rats.

(4) Ignition of CPP

24 h after the test on day 14, that was, on day 15, ignition was performed with a small amount of high-sugar and high-fat food, and the rats were placed in the middle box for a 15 min CPP value test.

Detection indicators: after the rats were trained, the conditioned place preference box was used to detect the addiction of high-sugar and high-fat food. A conditioned place preference score (CPP Score) reflects formation of the rat's addictive behavior. The increase of CPP Score indicates the formation of addictive behavior.

2. Experimental Results

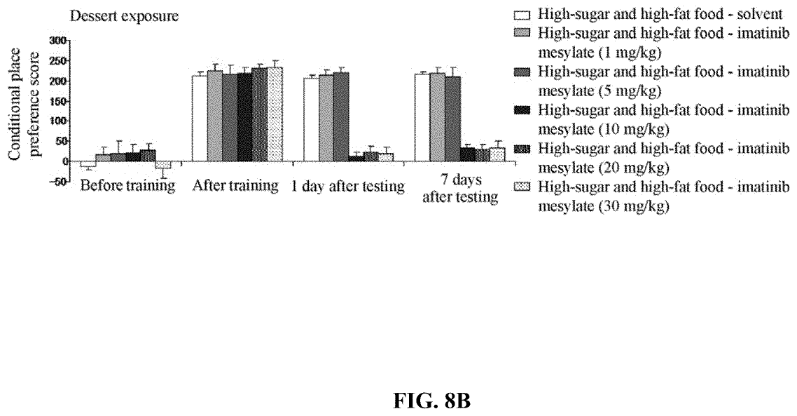

Results showed that the conditioned place preference was still present in the unmedicated rats; food-seeking behaviors induced by environment and food were inhibited after treatment with imatinib mesylate and were not ignited after 2 weeks. The results are shown in FIGS. 8A-8B. Differences between the different dose administration groups with the control group were significant, indicating that imatinib mesylate could improve the symptoms of high-sugar and high-fat food addiction and prevent relapse.

### Example 8

Inhibition of Conditioned Place Preference on High-Sugar and High-Fat Food in Rats Due to Environmental Re-Exposure or Unconditioned Re-Exposure by Administration of Imatinib Mesylate to Nucleus Accumbens

In this example, imatinib mesylate was selected as a drug against food addiction, a food-conditioned place preference (CPP) model was established, and an effect of imatinib mesylate on morphine reward memory was studied. An aim of this study was to select a drug with definite efficacy and low toxicity for the treatment of drug addiction.

1. Materials and Methods

Drugs and reagents: homemade high-sugar and high-fat food; imatinib mesylate (Novartis PharmaStein AG).

Experimental animals: SPF-grade SD male rats, weighing 220-250 g. The animals were provided by Hubei Provincial Laboratory Animal Research Center with an animal qualification certificate number of NO. 42000600012016 and a production license number of SCXK (Hubei) 2015-2018. Rat feed was purchased from the Laboratory Animal Center of Wuhan University.

Experimental apparatus: Conditional place preference instrument (developed by Institute of Materia Medica, Chinese Academy of Medical Sciences): the experiment was automatically controlled by a computer. The device was a conditioned place preference box consisting of three boxes: two side chambers and one middle chamber. The three chambers were separated by removable partitions, were black both inside and outside. Among them, box A and box B were located on two sides of the middle box, and had the same size. There were 9 squares formed by yellow light-emitting diodes on a side wall of box A, a bottom plate of box A was a stainless steel strip, and a bottom plate of box B was a stainless steel grid. The dwell time and number of entries and exits of the rats in each box could be transmitted to the computer via data, and behavioral information could be collected and recorded automatically.

Experimental Method

The rats were underwent localization surgery on the nucleus accumbens, and received CPP training with high-sugar and high-fat food one week later.

(1) Establishment of CPP Model for High-Sugar and High-Fat Food

Basal value test: on day 1, channels among the three boxes were opened, and a CPP program on the computer was started. The rats were put in from the middle chamber and allowed to move freely in the three boxes for 15 min. The time they stayed in each chamber was recorded via the computer synchronously.

Conditioned place preference training: on days 2 to 9, the channels among the three boxes were closed. On days 2, 4, 6 and 8, the experimental groups were given high-sugar and high-fat food freely and placed on the drug-paired side for 45 min; and the control groups were given cleared water and placed on the non drug-paired side for 45 min. On days 3, 5, 7 and 9, the rats in the experimental groups and the control groups were all given clear water, the experimental groups were placed on the non drug-paired side, and the control groups were placed on the drug-paired side, both for 45 min. The drug-paired side for each rat was fixed. Each group of rats was then returned to a rearing cage.

CPP test: CPP test was performed on day 10, which was similar to the basal value test phase. The channels among the three boxes were opened without any treatment, and the CPP program on the computer was started. The rats were put in from the middle chamber and allowed to move freely in the three boxes for 15 min. The time they stayed in each chamber was recorded via the computer synchronously. A CPP score was defined as a difference value between the time spent in the drug-paired chamber and the time spent in the non drug-paired chamber. Whether the rat developed CPP was determined by comparing a post-measurement value with a pre-measurement value for CPP of the rat in the drug-paired box. According to the post-measurement value for CPP, the rats that did not form CPP were excluded and the animals were matched and grouped.

(2) Establishment of a Model for Drug-Seeking Behavior Induced by Environmental Cues or Unconditioned Re-Exposure

On day 11 of the experiment, after exposure to the dosing box or administration of 5 g of high-sugar and high-fat food, the nucleus accumbens was microinjected with imatinib mesylate (4 μg/0.5 μL), and then the rats were returned to the cage environment.

(3) CPP Retest

On the first and seventh days after the administration of imatinib mesylate, that is, days 12 and 18 of the experiment, a preference degree of the rats to the drug-paired box was tested for 15 min, which was similar to the basal value test phase. On days 13-17 therebetween, no treatment was given to the rats.

(4) Ignition of CPP

24 h after the test on day 14, that was, on day 15, ignition was performed with a small amount of high-sugar and high-fat food, and the rats were placed in the middle box for a 15 min CPP value test.

Detection indicators: after the rats were trained, the conditioned place preference box was used to detect the addiction of high-sugar and high-fat food. A conditioned place preference score (CPP Score) reflects formation of the rat's addictive behavior. The increase of CPP Score indicates the formation of addictive behavior.

2. Experimental Results

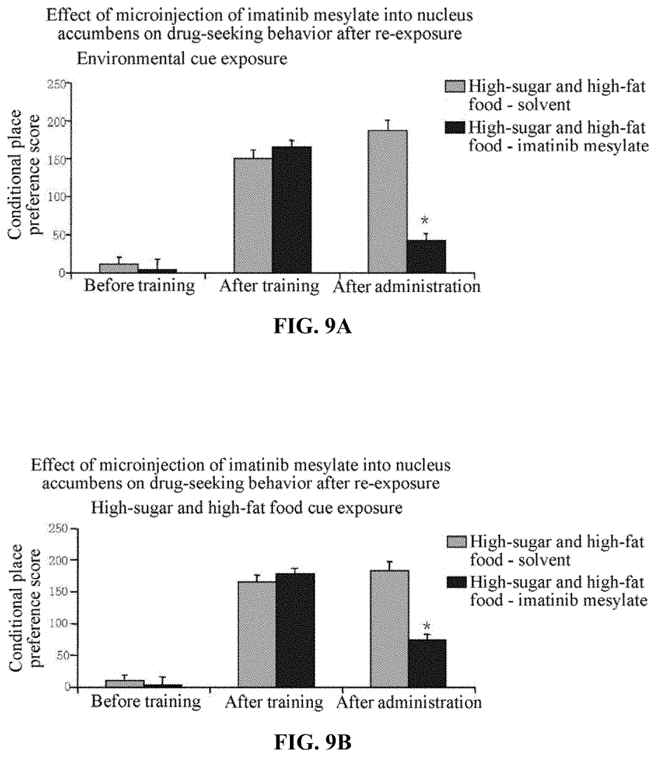

Results showed that the conditioned place preference was still present in the unmedicated rats; after the nucleus accumbens was administrated with imatinib mesylate, the food-seeking behavior caused by the environment and food was inhibited, and was not ignited after 2 weeks, which further confirmed the feasibility of c-Kit in the mesolimbic dopamine system as a therapeutic target for behavioral addiction drugs. The results are shown in FIGS. 9A-9B. A difference between the administration group and the control group was significant, indicating that imatinib mesylate could improve the symptoms of high-sugar and high-fat food addiction and prevent relapse.

### Example 9

Activation of c-kit activity in addiction-related brain regions under the condition of gambling addiction in rats and inhibition of formation of the condition of gambling addiction in rats by imatinib mesylate

1. Materials and Methods

Experimental animal: SPF-grade SD male rat, weighing 275-300 g, with an animal certificate number of NO. 42000600012016 and a production license number of SCXK (Hubei) 2015-2018. Rat feed was purchased from the Laboratory Animal Center of Wuhan University.

Experimental apparatus: five-well operating chamber, each operating chamber was enclosed in a ventilated sound-attenuating cabinet. Each chamber was provided with an array of 5 response wells 2 cm above a bar floor. There was a stimulation light behind each well. Nose-poke responses of these small wells were detected by a horizontal infrared beam. There were a food bank, an infrared beam and a tray light at the middle of an opposite wall, into which 45 mg of sucrose granules could be fed through an external granule dispenser. The chamber could be illuminated with room lights and controlled by software written by CAW in Med PC running on an ibm compatible computer.

Experimental Method:

A gambling behavior model of rats was established: animals were first accustomed to the operating chambers twice a day for 30 min each time, during which sucrose granules were placed on the reaction wells and the food banks. Animals were then trained to poke their noses into one of the luminescent reaction wells within 10 s to obtain a reward. Space positions of the stimulus light were varied in different experiments of wells 1, 2, 4 and 5. Each session consisted of 100 trials lasting approximately 30 min. After 5 trials, animals continued to complete 100 trials. Animals were then trained 7 times for forced-choice rGT (or an rGT variant of the control group), followed by a completely free-choice task, ensuring that all animals had the same experience in the four reinforcement conditions and were designed to prevent simple prejudice against specific wells. The percentage of animals selected for a particular option trial was calculated according to the formula (Journal of Illuminated Food). Each experiment was 30 min in a 3-day cycle, and the baseline was measured on the first day; on the second day, the rats received drug or saline injections 30 min before the test; on the third day, the animals were not tested and were injected with imatinib mesylate 30 min before the start of behavioral testing.

After the test, immunohistochemistry was used to observe changes of c-Kit activity in the mesolimbic dopamine system including VTA, nucleus accumbens, amygdala, hippocampus, prefrontal cortex and cerebellar peduncle after formation of gambling behavior in each group of rats, and the effect of imatinib mesylate on c-Kit phosphorylation levels to determine the role of c-Kit in gambling behavioral addiction.

2. Experimental Results

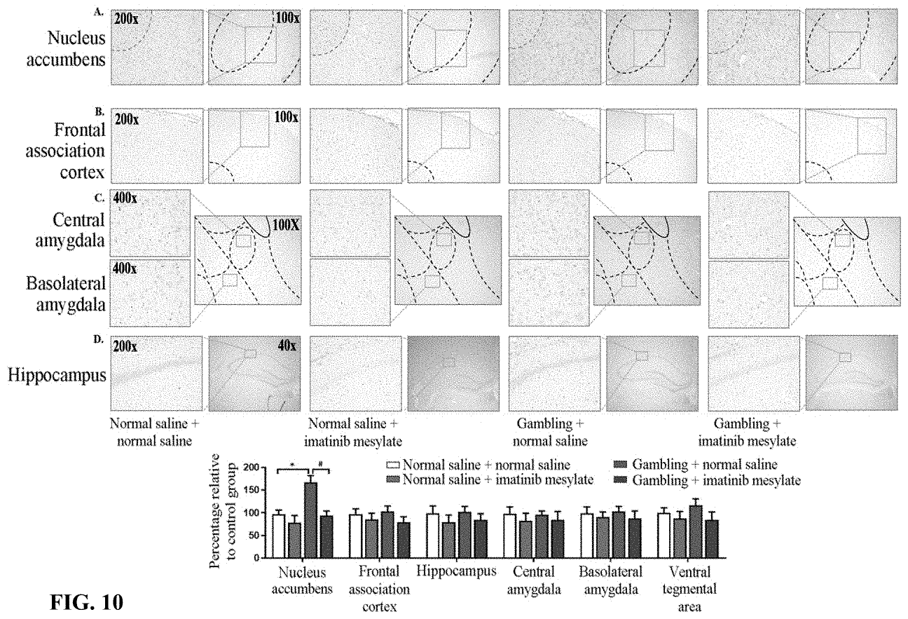

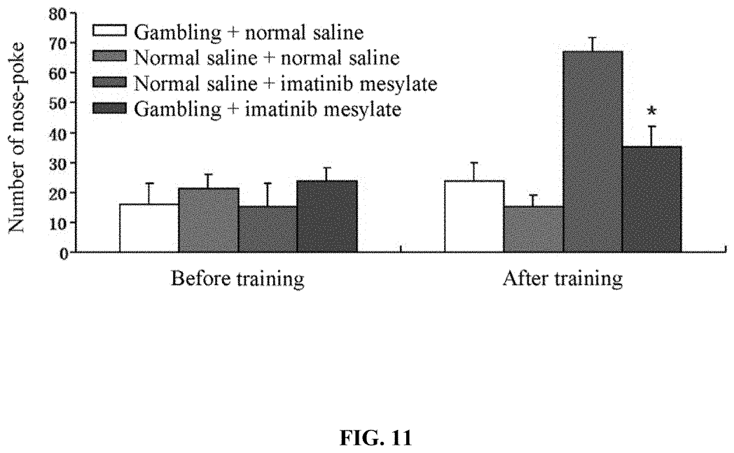

Results showed that gambling behavior caused different degrees of changes in the c-Kit activity in the mesolimbic dopamine system including VTA, nucleus accumbens, amygdala, hippocampus, prefrontal cortex and cerebellar peduncle, in particular, the c-Kit activity in the nucleus accumbens was significantly enhanced. Administration of imatinib mesylate (30 mg/kg) significantly inhibited the phosphorylation level of c-Kit (see FIG. 10 for the results), and significantly eliminated gambling behavior (see FIG. 11 for the results), indicating the core role of c-Kit in gambling behavioral addiction and therapeutic effects of imatinib mesylate.

### Example 10

Dose Effect of Imatinib Mesylate on Gambling Behavior in Rats Under Gambling Task Conditions

1. Materials

Experimental animals: SPF-grade SD male rats, weighing 275-300 g. The animals were provided by Hubei Provincial Laboratory Animal Research Center with an animal qualification certificate number of NO. 42010200001574 and a production license number of SCXK (Hubei) 2017-0012. Rat feed was purchased from the Laboratory Animal Center of Wuhan University.

Experimental apparatus: five-well operating chamber, each operating chamber was enclosed in a ventilated sound-attenuating cabinet. 5 arrayed response wells were placed 2 cm above a bottom of each operating chamber, and a stimulation light was placed behind each well. Nose-poke responses of these small wells could be detected with a horizontal infrared beam. There were a food bank, an infrared beam and a tray light at the middle of an opposite wall, into which 45 mg of sucrose granules could be fed through an external granule dispenser. The chamber could be illuminated with room lights and controlled by software written by CAW in Med PC running on an IBM compatible computer.

2. Experimental Method:

Experimental animal grouping: a total of 6 groups (n=10), namely a normal saline group, and 1, 5, 10, 20 and 30 mg/kg of imatinib mesylate groups, totally 6 groups.

A gambling behavior model of rats was established: firstly, the animals were acclimated to the operating chamber twice a day for 30 min each time, during which sucrose granules were placed on the reaction wells and the food bank. After acclimation, animals were trained to poke their noses into one of the luminescent reaction wells within 10 s to obtain a reward. Spatial positions of the stimulus light would appear in different wells of wells 1, 2, 4 and 5 in different experiments. Each session consisted of 100 trials lasting approximately 30 min. Animals were then trained 7 times for forced-choice rGT (or an rGT variant of the control group), and then underwent a completely free-choice task. This ensured that all animals had the same experience under the four reinforcement conditions and were designed to prevent simple prejudice against specific wells. The percentage of trials in which animals chose a particular option was calculated according to the formula in reference: Choices of a particular option/Total choices of 100 (Di C P, Manvich D F, Pushparaj A, et al. Effects of disulfiram on choice behavior in a rodent gambling task: association with catecholamine levels[J]. Psychopharmacology, 2018, 235(1):23-35). Each experiment lasted 30 min and experimental subjects responded with a nose poke on the illuminated food bank, which turned off the tray light and triggered the initiation of a 5-s intertrial interval (ITT). At the end of the ITI, wells 1, 2, 4 and 5 were illuminated for 10 s (in the forced-choice version of the task used in the training, only one well was illuminated). If the animal did not respond within 10 s, the trial would be marked as missed, at which point the tray light would be turned on again and the animal could start a new trial.

| Option 1 | Option 2 | Option 3 | Option 4 |
|---|---|---|---|
| 1 sucrose | 2 sucrose | 3 sucrose | 4 sucrose |
| granule, | granules, | granules, | granules, |
| p = 0.9 | p = 0.8 | p = 0.5 | p = 0.4 |
| 5 s | 10 s | 30 s | 40 s |
| penalty time, | penalty time, | penalty time, | penalty time, |
| p = 0.1 | p = 0.2 | p = 0.5 | p = 0.6 |

Explanation to the above table: four wells in the experimental apparatus were respectively provided with different reward probability and penalty probability, as well as corresponding reward sucrose amount and penalty time. There was no food reward during the penalty time, indicating that after completing a series of choices within 30 min, choosing only option 2 will get the best benefit.

The rats were trained until the baseline of the rats was stable, and the rats in the rGT group always showed a preference for the choice of two sucrose granules, that was, the best benefit choice; the overall tendency was P2>P4>P1>P3, but in fact, the best options were ranked as P2>P1>P3>P4.

After training, the rats were given with drugs. On the first day after the baseline was stable, the rats induced by environmental cues were put into the experimental device as in the acclimation period, but the experiment was not started, and then given with imatinib mesylate (1, 5, 10, 20 and 30 mg/kg, ip) or saline (1 mL/kg, ip). The rats in the direct administration group were directly given with imatinib (1, 5, 10, 20 and 30 mg/kg, ip) or saline (1 mL/kg, ip) without being placed in the experimental device. After that, all the rats were put back into their cages, and behavioral testing was performed on day 1 after administration. On day 7 after administration, behavioral testing was performed again.

3. Experimental Results

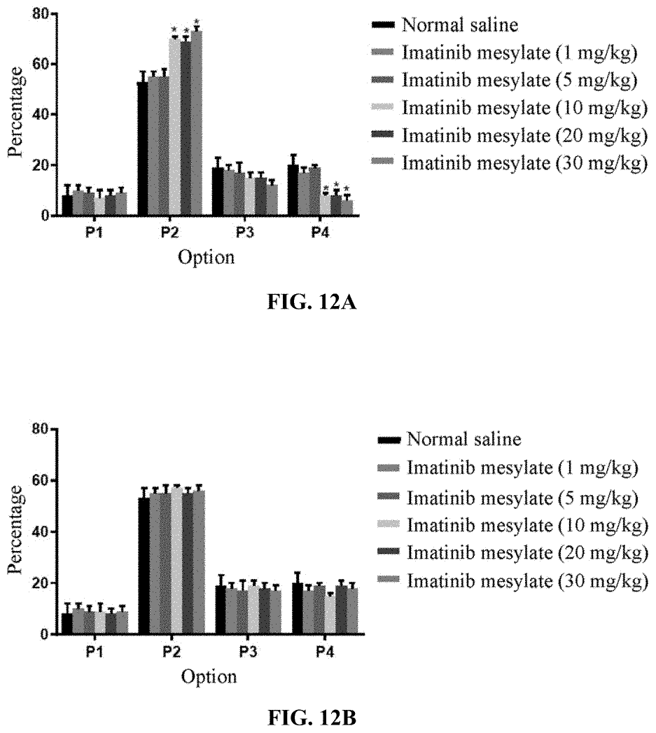

Results are shown in FIGS. 12A-12B: for the rats exposed to the environment, 10, 20 and 30 mg/kg of imatinib mesylate significantly increased the optimal choice at P2 and reduced the choice at P4 in rGT after administration of imatinib mesylate compared to the control group; however, for the rats as directly administered, no significant effect of imatinib mesylate was found at any dose. The results showed that, for the gambling task in rats, after baseline stabilization, giving animals to environmental cues induction prior to administration could enhance the improvement effects compared to the direct administration.

Conclusion: behavioral addictions such as high-sugar and high-fat food or gambling activate c-Kit receptors in nucleus accumbens neurons, and systemic administration of imatinib mesylate can inhibit c-Kit receptor activity and inhibit the formation of conditioned place preference and gambling behavior. Meanwhile, systemic administration or microinjection of imatinib mesylate in the nucleus accumbens can inhibit food-seeking behaviors elicited by environmental cues and food, indicating that c-Kit receptors play a key role in food behavioral addiction, and designing drugs to inhibit its activity can achieve the effect of inhibiting the formation of behavioral addiction or post-addiction prevention and relapse prevention.

It will be obvious to those skilled in the art that changes and modifications may be made, and therefore, the aim in the appended claims is to cover all such changes and modifications.

What is claimed is:

1. A method of diagnosing and monitoring substance addiction or behavioral addiction, the method comprising using a biomarker, wherein the biomarker is a biomarker produced by c-Kit gene.

2. A product for reflecting a substance or behavioral addiction state by tracing or detecting and monitoring various RNA, DNA or c-Kit protein activities and related metabolites of c-Kit in an addicted patient.

3. The product of claim 2, wherein the substance comprises narcotic drugs, psychotropic drugs, alcohol, tobacco, and volatile organic solvents; the narcotic drugs comprise opioids, cocaine, cannabis and other drugs; the psychotropic drugs comprise sedative-hypnotics, anxiolytics, central stimulants, hallucinogens and the like; and the behaviors comprise internet addiction, gambling addiction and other behaviors.

4. The product of claim 2, wherein the product comprises test strips, kits, chips, high-throughput sequencing platforms or imaging and other in vivo or in vitro diagnostic products.

5. The product of claim 2, wherein the product is capable of diagnosing and monitoring substance addiction and behavioral addiction by detecting expression of c-Kit gene, RNA and protein in a sample or related metabolites thereof; and the sample comprises blood, urine, saliva and other bodily fluids and tissues.

6. The product of claim 2, wherein a purpose of detecting activities of c-Kit gene, RNA and protein is achieved by the product based on various methods comprising reverse transcription PCR, fluorescent real-time quantitative PCR, immunoassay, in-situ hybridization, chip, high-throughput sequencing platform or brain functional magnetic resonance and omics.

7. A method for screening a drug for treating behavioral addiction comprising using c-Kit as a behavioral addiction treatment target.

8. The method of claim 7, wherein the behavioral addiction comprises gambling, eating, sexual behavior, Internet, work, exercise, mental compulsion and shopping addictions.

9. The method of claim 7, wherein the drug for treating behavioral addiction exhibits an inhibitory effect on c-Kit.

10. The method of claim 9, wherein the drug for treating behavioral addiction is imatinib or a derivative thereof.
